(19) United States
(12) Patent Application Publication
(10) Pub. No.: US 2018/0071414 A1
(43) Pub. Date: Mar. 15, 2018

(54) UV-C Based Skin Sterilization Device

(71) Applicants: Mauricio Dujowich, Newberry, FL (US); Jennifer Jean Bentley, Newberry, FL (US)

(72) Inventors: Mauricio Dujowich, Newberry, FL (US); Jennifer Jean Bentley, Newberry, FL (US)

(73) Assignee: One Health Labs, Inc., Newberry, FL (US)

(21) Appl. No.: 15/300,744

(22) PCT Filed: May 3, 2016
(86) PCT No.: PCT/US2016/030608

§ 371 Date: Sep. 29, 2016

Related U.S. Application Data
(60) Provisional application No. 62/156,361, filed on May 4, 2015.

(51) Int. Cl. A61L 2/00; A61L 2/24

(52) U.S. Cl. CPC A61L 2/0047; A61L 2202/14; A61L 2202/11; A61L 2/24

(57) ABSTRACT

Disclosed is a UV-C based skin sterilizer that includes a main console connected to a delivery system and a display screen, the delivery system including a dosing system and a UV-C light source, wherein the UV-C light source can be an LED array or mercury or xenon lamps. The dosing system includes an image recognition module that is in communication with a camera, a light absorption sensor, a distance sensor, and an accelerometer/speed sensor. The dosing system communicates with a microcontroller of the main console in order to deliver a three-dimensional mapping of a treatment area and to optimize the dose of UV-C being delivered for the purposes of sterilizing the treatment area. The display screen provides a user interface for viewing the three-dimensional mapping and to receive user input.

### CROSS-REFERENCE TO RELATED APPLICATIONS

This application claims the benefit of U.S. Provisional Patent Application No. 62/156,361, filed May 4, 2015, which is hereby incorporated by reference in its entirety.

### FIELD OF THE INVENTION

The present invention relates generally to a skin sterilization device and method of use. More particularly, the present invention is directed to an ultraviolet-C (UV-C) based skin sterilization device for reducing the bacterial burden on skin incisions or wound surfaces.

### BACKGROUND OF THE INVENTION

Ultraviolet germicidal irradiation (UVGI) is a disinfection method that uses ultraviolet (UV) light at sufficiently short wavelengths to kill microorganisms such as bacteria. It is used in a variety of applications, such as food, air, and water purification. UVGI utilizes UV-C that is harmful to microorganisms. It is effective in destroying the nucleic acids in these organisms so that their DNA is disrupted by the UV radiation, leaving them unable to perform vital cellular functions.

UVGI is primarily used for air sanitation and water purification. Germicidal UV may be delivered by a mercury-vapor lamp that emits UV at the germicidal wavelength. Mercury-vapor lamps and other UV lamps are generally static in nature and are not optimized to deliver appropriate doses based on various parameters during treatment. In this regard, existing devices may be unable to deliver an optimal dose of UV-C for the purposes of sterilizing skin. Thus, a device that is optimized to deliver a safe and more precise dose of UV-C to sterilize skin incisions or wound surfaces is desired. In this regard, the invention described herein addresses this problem.

### SUMMARY OF THE INVENTION

The following discloses a simplified summary of the specification in order to provide a basic understanding of some aspects of the specification. This summary is not an extensive overview of the specification. It is intended to neither identify key or critical elements of the specification nor delineate the scope of the specification. Its sole purpose is to disclose some concepts of the specification in a simplified form as to prelude to the more detailed description that is disclosed later.

One embodiment of the present invention provides a UV-C based skin sterilization device that is adapted to deliver an appropriate dosage of UV-C to a designated treatment area. The device comprises a main console, a delivery system, and a display output (e.g., display screen), wherein the main console and the delivery system are connected via an electrical conduit and/or fiber optics.

The main console comprises a microcontroller in communication with a UV-C lamp, a timer, a speaker, a projector, and a wireless module. The main console is further connected to a display screen, a trigger, and a power supply.

The delivery system comprises a dosing system and a light source, wherein the light source comprises UV-C LED array or another UV-C light source such as a UV-C lamp. The dosing system includes a camera, a distance sensor, a light absorption sensor, an accelerometer/speed sensor, and image recognition module that can project a three-dimensional representation of the treatment site. Further, the image of the treatment site (i.e., on the display screen) can be divided into a grid using, for example, lasers or other image projecting means, so as to allow the user to easily distinguish treated areas from untreated areas. During operation, the microcontroller is configured to continuously control the dosage of UV-C delivered via the UV-C LED array while monitoring the amount of UV-C delivered at the treatment sites by communicating with the sensors described herein.

It is therefore an objective of the present invention to provide a sterilization device that utilizes germicidal UV-C technology.

It is still another objective of the present invention to provide a sterilization device that is configured to automatically deliver a safe and precise dose of UV-C.

It is still another objective of the present invention to provide a sterilization device that continuously monitors a treatment site in order to deliver an effective dosage of UV-C while in use.

It is still another objective of the present invention to provide a delivery and dosing system that can be applied to a wide variety of light emitting devices that require accurate dosing for use on a variety of surfaces.

Another objective of the present invention is to provide a UV-C based skin sterilizer that may be readily fabricated from materials that permit relative economy and commensurate with durability.

In the light of the foregoing, these and other objectives are accomplished in accordance of the principles of the present invention, wherein the novelty of the present invention will become apparent from the following detailed description and appended claims.

### BRIEF DESCRIPTION OF THE DRAWINGS

The above and other objects and advantages of the present invention will be apparent upon consideration of the following detailed description, taken in conjunction with the accompanying exemplary drawings, in which like reference characters refer to like parts throughout, and in which:

FIG. 1 is a high-level block diagram of an electronic device, in accordance with an example implementation.

FIG. 2 shows exemplary method steps of the present method.

FIG. 3 shows exemplary method steps for defining a treatment site.

FIGS. 4A and 4B show exemplary treatment steps of the present invention.

FIG. 5 shows exemplary three-dimensional mapping of the present invention.

### DETAILED DESCRIPTION OF THE INVENTION

The present invention is directed towards a UV-C based skin sterilization device. For purposes of clarity, and not by way of limitation, illustrative views of the present device are described with references made to the above-identified figures. Various modifications obvious to one skilled in the art are deemed to be within the spirit and scope of the present invention.

As used in this application, the terms “component,” “module,” “system,” “interface,” or the like are generally intended to refer to a computer-related entity, either hardware or a combination of hardware and software. For example, a component can be, but is not limited to being, a process running on a processor, an object, and/or a computer. By way of illustration, both an application running on a controller and the controller can be a component. One or more components can reside within a process and/or thread of execution and a component can be localized on one computer and/or distributed between two or more computers. As another example, an interface can include I/O components as well as associated processor, application, and/or API components.

Furthermore, the claimed subject matter can be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, or media. It is to be appreciated that determinations or inferences referenced throughout the subject specification can be practiced through the use of artificial intelligence techniques.

Some embodiments may be used in conjunction with various devices and systems, for example, a personal computer (PC), a desktop computer, a mobile computer, a laptop, a tablet computer, a server computer, a handheld device, a personal digital assistant (PDA), a wireless communication device, a smart phone, a non-portable device, a wireless access point (AP), a wired or wireless router, a wired or wireless modem, a wired or wireless network, a local area network (LAN), a wireless LAN (WLAN), a metropolitan area network (MAN), a wireless MAN (WMAN), a wide area network (WAN), a wireless WAN (WWAN), a personal area network (PAN), a wireless PAN (WPAN), or networks operating in accordance with existing and/or future versions and/or derivatives of long term evolution (LTE), a device which incorporates a global positioning system (GPS) receiver or transceiver or chip, a device which incorporates an RFID element or chip, a multiple input multiple output (MIMO) transceiver or device, a single input multiple output (SIMO) transceiver or device, a multiple input single output (MISO) transceiver or device, a device having one or more internal antennas and/or external antennas, or the like.

Moreover, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to disclose concepts in a concrete fashion. As used in this application, the term “or” is intended to mean an inclusive “or” rather than an exclusive “or.” The articles “a” and “an” as used in this application and the appended claims should generally be construed to mean “one or more” or “at least one” unless specified otherwise or clear from context to be directed to a singular form. Additionally, as used herein, the terms “treatment surface,” “treatment area,” “treatment areas,” “treatment site,” “treatment sites,” “area,” “areas,” “wound,” “skin,” and “skin wound” may be used interchangeably unless the context clearly suggests otherwise, wherein the foregoing terms mean any surface that can be treated with light emitting devices having a delivery or dosing system.

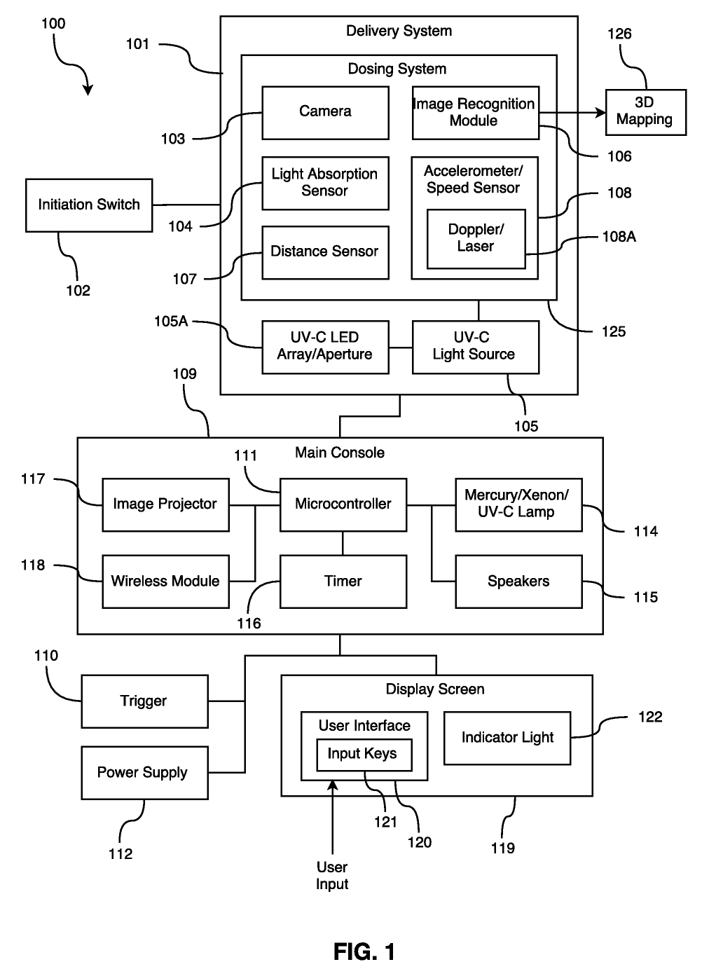

Referring now to FIG. 1 there is shown an exemplary schematic diagram of the present invention. The present sterilization device 100 comprises a main console 109, a delivery system 101, and a display output (e.g., display screen 119), wherein the main console 109 and the delivery system 101 are connected via an electrical conduit and/or fiber optics such that it is relatively light weight and improved in reliability. Alternatively, the connectivity may also be accomplished entirely via the application of wireless technology.

The main console 109 comprises a microcontroller 111 or a processor that is coupled to a memory unit. The microcontroller 111 comprises a main processing unit or processor, one or more memory units (e.g., RAM, ROM), and input/output (I/O) ports. In the illustrated embodiment, the microcontroller 111 is connected to speakers 115, an image projector 117, a wireless module 118, a timer 116, and a UV-C lamp 114.

In other embodiments, the main console 109 may comprise other input devices and output devices for receiving information from external sources and for transmitting information externally. For example, some embodiments can comprise a secondary or an additional lighting apparatus that may illuminate the skin (i.e., the treatment surface) to provide an improved visibility of the same. Additionally, in some embodiments, the present invention may comprise memory units that are located remotely (e.g., databases in a network).

The main console 109 further comprises a trigger 110 or a power switch that is connected to an external or an internal power supply 112 (e.g., batteries or a power adaptor that can be connected to an outlet). The power supply 112 delivers power, upon receiving signal from the trigger 110, to the main console 109, which is connected to the delivery system 101 and the display screen 119.

The main console 109 is connected to a display screen 119, wherein the display screen 119 can be an external display screen (e.g., a monitor) that is connected or made integral to the main console 109 such that it is mounted on the exterior of the main console 109. The display screen 119 preferably comprises a touch screen having a user interface 120 for receiving user input. The user interface 120 facilitates communication between a user of the device 100 and one or more elements of the present invention.

The user interface 120 may thus be configured to allow users to enter commands, for example, via virtual input keys 121 or buttons thereon. It is contemplated that in some embodiments, the device 100 further comprises other control buttons for utilizing the same. The user interface 120 further provides notification features to notify the user with various alerts such as the battery level and treatment progress, among others.

The delivery system 101 comprises a dosing system 125 and a UV-C light source 105 (e.g., UV-C LED array/aperture 105A, mercury/xenon lamp). The UV-C LED array/aperture 105A may be remotely connected to the main console 109 or a UV-C light source 105 may be directly connected to a different type of UV-C lamp 114. In this regard, some embodiments of the device 100 without the UV-C LED array/aperture 105A comprise a UV-C lamp 114 with a protective covering (i.e., with perforations for emitting light therethrough) on the main console 109 that remains securely closed until the device 100 is activated and has stable power. Conversely, some embodiments of the main console 109 does not require a UV-C lamp 114 when the dosing system 125 comprises UV-C LED array/aperture 105A.

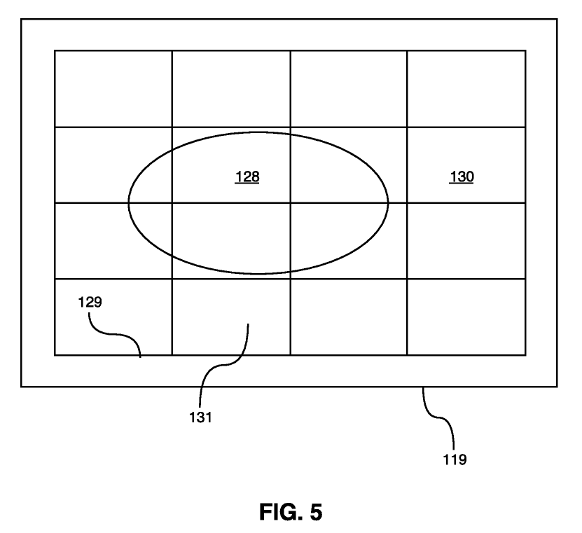

The dosing system 125 comprises at least one camera 103, a distance sensor 107, a light absorption sensor 104, an accelerometer/speed sensor 108 having a mounted Doppler/laser 108A, and image recognition module 106 that is adapted to perform three-dimensional mapping 126 of the surface to be treated or being treated. The image recognition module 106 obtains captured images of the treatment surface from the camera 103 so as to automatically recommend treatment boundaries for the user, which the user can accept or manually modify using the input or control keys 121 on the user interface 120 and/or the touch screen. In this regard, the images 130 (FIG. 5) captured by the camera 103 can be viewed on the display screen 119, preferably in real-time (e.g., via video feed), as depicted in FIG. 5.

Three-dimensional mapping 126 of the treatment area 128 (FIG. 5) includes images 130 (FIG. 5) of the treatment area 128 (FIG. 5) that are divided into segments or that includes overlaid grid 129 (FIG. 5) to facilitate treatment. The image recognition module 106 is further configured to change the color of each area 131 (FIG. 5) of the grid 129 (FIG. 5) on the display screen 119 during treatment sessions to differentiate areas that are treated and untreated, or to show that appropriate dose of UV-C is delivered to the treatment area 128.

The image recognition module 106 is further configured to determine the dose delivery at the surface via the light absorption sensor 104, which is configured to measure the amount of light wave that is absorbed by the skin to ensure that the treatment area is absorbing the targeted amount of UV-C. The image recognition module 106 further communicates with the distance sensor 107, which measures the distance between the device 100 (i.e., the UV-C light source 105) and the treatment surface to determine whether the device 100 is positioned at a desired distance relative to the treatment area.

Similarly, the accelerometer/speed sensor 108 is configured to measure the speed and acceleration at which the device 100 (i.e., the UV-C light source 105) is being moved in order to determine whether the speed or acceleration is within the desired range. In this regard, the accelerometer/speed sensor 108 works in conjunction with a mounted Doppler or lasers 108A with respective receivers to calculate speed. In this way, the accelerometer/speed sensor 108 is used to ensure that the treatment site receives sufficient exposure to UV-C during treatment.

It is contemplated that the targeted or the predetermined ranges of light absorption, distance, acceleration, and/or speed are preprogrammed and stored in the microcontroller 111 and/or another memory unit. When the measured light absorption, distance, acceleration, and/or speed are below or beyond the respective predetermined ranges, the microcontroller 111 may be configured to signal one or more output devices to emit notifications to the user. For instance, the speakers 115 may be configured to emit beeps. Additionally, the user interface 120 can display textual/pictorial notifications. Upon determining that light absorption, distance, acceleration, and/or speed are within the respective predetermined ranges, for example, after proper adjustments are made via the microcontroller 111, the notifications can automatically stop.

The trigger 110 is used to activate the device. When the trigger 110 is depressed, the timer 116 counts down before the device 100 is activated so as to give time to a user to be ready to begin administering treatment. Alternatively, the device 100 may be activated for use via an on/off switch. Additionally, it is contemplated that the delivery system 101 comprises a separate initiation switch 102 for activating the same, depending upon embodiment.

Once the device 100 is activated, the speakers 115 and/or the indicator light 122 on the display screen 119 can emit signals to indicate that the device 100 is turned on. For instance, the speakers 115 can emit beeps at regular intervals and/or the indicator light 122 can illuminate. It is contemplated that the speakers 115 and the indicator light 122 remains activated while the device 100 is turned on. If the device 100 is idle for a predetermined period of time after it has been activated and senses no motion, the device 100 can automatically turn itself off or enter into a power saving mode.

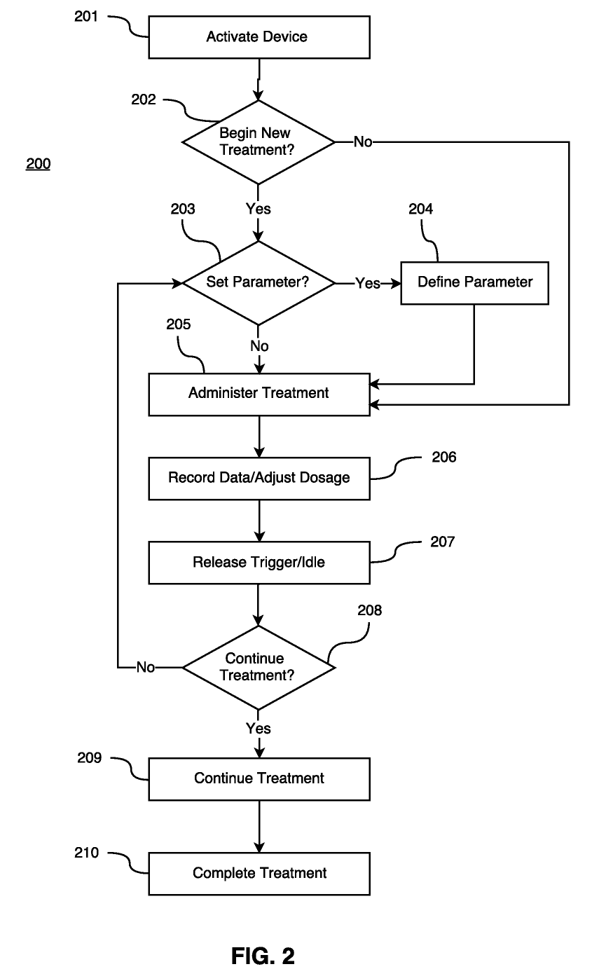

Reference is also made to FIG. 2, which schematically illustrates a method of the present invention. One or more of the operations of FIG. 2 may be performed by one or more elements of the present invention as illustrated in FIG. 1. As indicated in block 201, the device 100 (FIG. 1) is activated, for example, via the trigger 110 (FIG. 1) and/or an on/off switch. When the device 100 (FIG. 1) is activated, a user is prompted, via the user interface 120 (FIG. 1) to continue an existing treatment or begin new treatment 202.

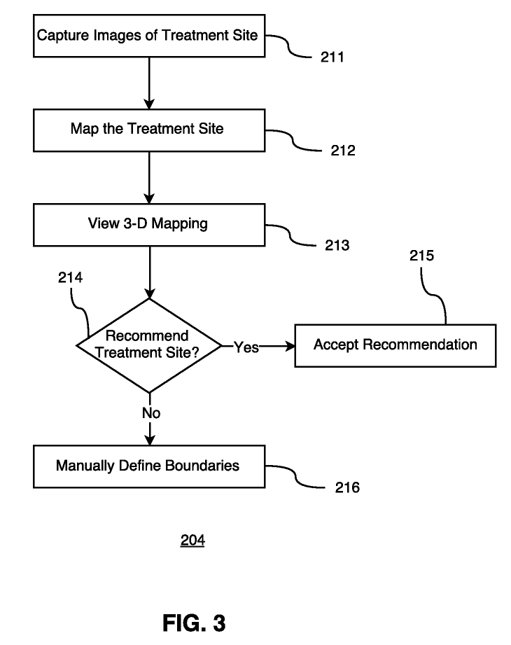

To begin a new session for a treatment, the user user is prompted to set parameters for the treatment as indicated in block 203. In this regard, the user can define parameters 204 for that specific treatment session. Without limitation, treatment parameters can include treatment type, duration, and area/site, among others. In one embodiment, the user can define the proposed treatment site and create treatment boundaries so that UV-C is delivered only to the desired treatment site, as indicated in FIG. 3.

As indicated in block 211, the present method includes capturing images of the treatment site, for example, via the camera 103 (FIG. 1). The images are displayed on the display screen 119 (FIG. 1) and used to map the treatment site 212 and produce a three-dimensional mapping of the treatment area. As indicated in block 213, the three-dimensional mapping can be displayed and viewed on the display screen 119 (FIG. 1). As indicated in block 214, the image recognition module 106 (FIG. 1) can automatically recommend the proposed treatment site using the three-dimensional mapping 216 (FIG. 1). The user can accept the recommendation 215 or manually define the treatment boundaries 216 by using the user interface 120 (FIG. 1). It is contemplated, however, that the user can bypass setting treatment boundaries and begin treatment without defining the treatment boundaries.

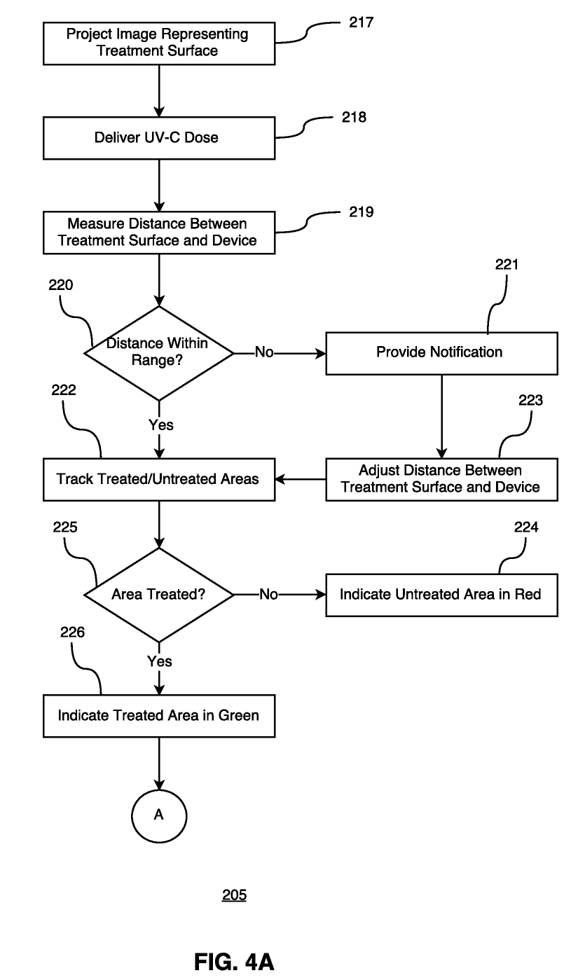

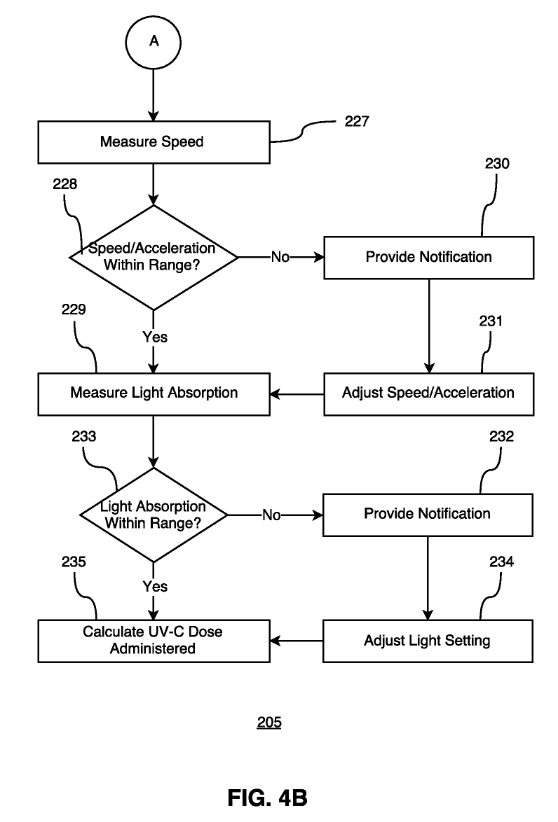

As indicated in block 205, the user can begin administering treatment after the parameters are defined. Detailed method steps of administering treatment are depicted in FIGS. 4A and 4B. As indicated in block 217, the present method includes projecting an image showing the representation of the surface being treated, via, for example, the image projector 117 (FIG. 1). The image can be projected directly onto the surface area or the display screen 119 (FIG. 1), wherein the display screen 119 (FIG. 1) can be an external device such as a monitor or a similar display device, further wherein the image data is transmitted via the wireless module 118 (FIG. 1). Additionally, the image 130 (FIG. 5) of the treatment area 128 (FIG. 5) can be divided into a grid configuration 129 (FIG. 5).

As indicated in block 218, the UV-C light source 105 (FIG. 1) is activated to deliver UV-C dose. While the device is in use, the distance sensor 107 (FIG. 1) continuously measures the distance between the treatment surface and the device 100 (FIG. 1) to determine whether the distance is within a predetermined range 220. If the measured distance is not within the desired range, the microcontroller 111 (FIG. 1) is configured to signal the display screen 119 (FIG. 1) and/or the speakers 115 (FIG. 1) to provide a notification 221 on the user interface 120 (FIG. 1) and/or to emit audible alerts, respectively. Upon receiving the notification, the user can adjust the distance between the treatment surface and the device 223. When the distance is within the predetermined range, the notification automatically stops. In another embodiment, the power output of the UV-C light source 105 (FIG. 1) may be modified as necessary to deliver an appropriate or a predetermined total dose.

As indicated in block 222, the method further includes tracking treated and untreated areas, for example, via the image recognition module 106 (FIG. 1). As indicated in blocks 224, 226, each area of the grid can then change in color as they are treated, or as appropriate dose of UV-C is delivered to the area. The color change corresponds to the dose delivery and the movement of the user in real-time. In one embodiment, treated areas can be represented in green 226 and untreated areas can be represented in red 224. In another embodiment, the area being treated may be represented in yellow, or there may be a gradient change corresponding to the amount of dose delivered.

If the user attempts to treat a grid area that has already turned green, (i.e., indicating that the treatment site was treated), the UV-C LED array 105A (FIG. 1) or another UV-C light source 105 (FIG. 1) can automatically deactivate, and then reactivate when the user moves onto a grid area that is red and that has not been fully treated. Similarly, when the user attempts to deliver UV-C onto an area outside of the treatment boundary, the UV-C LED array 105A (FIG. 1) or another UV-C light source 105 (FIG. 1) can deactivate, and then reactivate once the user moves back within the defined treatment area so long as the dose end point has not been reached.

As indicated in block 227, the method further includes measuring the speed and acceleration of the device 100 (FIG. 1), via the accelerometer/speed sensor 108 (FIG. 1) and mounted Doppler or lasers 108A (FIG. 1) with respective receivers for calculating speed, to determine if the speed or the acceleration is within a desired range 228. If the measured speed or the acceleration is not within the desired range, the microcontroller 111 (FIG. 1) is configured to signal the display screen 119 (FIG. 1) and/or the speakers 115 (FIG. 1) to provide a notification 230 on the user interface 120 (FIG. 1) and/or to emit audible alerts, respectively. Upon receiving the notification, the user can adjust the speed or acceleration at which the device moves 231. When the speed or acceleration is within the predetermined range, the notification automatically stops.

As indicated in block 229, the method further includes measuring the light absorption on the treatment surface, via the light absorption sensor 104 (FIG. 1) to determine whether the device 100 is delivering the appropriate dosage of UV-C. If the measured light absorption is not within a predetermined range 233, the microcontroller 111 (FIG. 1) is configured to signal the display screen 119 (FIG. 1) and/or the speakers 115 (FIG. 1) to provide a notification 232 on the user interface 120 (FIG. 1) and/or to emit audible alerts, respectively. Upon receiving the notification, the device's light settings of the UV-C light source 105 (FIG. 1) can be automatically adjusted 234 via the microcontroller 111 (FIG. 1). When the light absorption is within the predetermined range, the notification automatically stops.

It is noted that the steps as indicated in blocks 219 through 234 as shown in FIGS. 4A and 4B can occur in any order and/or concurrently, depending upon embodiment. Additionally, the steps as indicated in blocks 219 through 234 are not necessarily dependent upon one another. For instance, the present method may include the steps of measuring the device's speed without measuring the light absorption. Additionally, it is contemplated that the image recognition module 106 (FIG. 1), the light absorption sensor 104 (FIG. 1), the accelerometer/speed sensor 108 (FIG. 1), and/or the distance sensor 107 (FIG. 1) are configured to continuously make measurements to deliver optimized dose of UV-C while the device 100 (FIG. 1) is in use.

As the user moves the device 100 (FIG. 1) over the treatment area, the microcontroller 111 (FIG. 1) is adapted to continuously adjust the dosage of UV-C based on the distance, light absorption, location, acceleration, and speed, wherein the microcontroller 111 (FIG. 1) can retrieve and receive data from the foregoing sensors (i.e., the light absorption sensor, the accelerometer/speed sensor, the distance sensor) to calculate the actual dosage given 235.

As indicated in block 206, the microcontroller 111 (FIG. 1) is configured to record data and adjust dosage. More specifically, the microcontroller 111 (FIG. 1) is configured to transmit data to an external device (e.g., a computer system, an electronic device) during treatment so as to allow the user to understand the status of each treatment. More specifically, the measurements obtained from the sensors (i.e., the light absorption sensor, the accelerometer/speed sensor, the distance sensor) and the administered dosages, along with other information associated with or related to each of the treatment sessions are automatically recorded and transmitted to the external computer system (e.g., a database) or the main console 109 (FIG. 1) during treatment so that the user is aware of the status of each treatment.

In some embodiments, the microcontroller 111 (FIG. 1) can automatically adjust the dosage by controlling the UV-C LED array 105A (FIG. 1) or the UV-C light source 105 (FIG. 1) (e.g., UV-C lamp) using the treatment data. For instance, the microcontroller 111 (FIG. 1) can activate some or all of the LEDs. Further, the microcontroller 111 (FIG. 1) can detect the power output of the UV-C LED arrays so that it can calculate dosing.

When the trigger 110 (FIG. 1) is released during the treatment or the device 100 (FIG. 1) becomes idle 207, the UV-C LED array 105A (FIG. 1) can deactivate. If the user depresses the trigger 110 (FIG. 1) after releasing it, the user will be prompted to continue with the current session or start a new session 208. Alternatively, the device 100 (FIG. 1) can automatically start a new session if a predetermined amount of time has elapsed., wherein the amount of time that has elapsed can be measured via the timer 116 (FIG. 1). If the user selects to continue treatment, the settings and parameters used during the session would be restored or retrieved from a memory unit and the user can continue treatment 209 until the treatment is completed 210. For instance, all of the treated areas will remain marked as treated areas. If the user selects to start a new session, the user will be prompted to set new parameters such as the boundary of a new treatment site.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A UV-C based sterilization device, comprising:
a delivery system comprising a dosing system and a UV-C light source;
said dosing system comprising an image recognition module in communication with a camera, wherein said image recognition module is configured to produce a three-dimensional mapping of a treatment surface;
a main console comprising a microcontroller configured to:
calculate a dose of UV-C being delivered to said treatment surface;
adjust said dose of UV-C to optimize said dose of UV-C being delivered to said treatment surface; and
a display screen connected to said main console, wherein said display screen comprises a user interface for receiving user input.

2. The UV-C based sterilization device of claim 1, wherein said dosing system further comprises a light absorption sensor;
said light absorption sensor configured to measure amount of light absorbed at said treatment surface;
wherein said microcontroller is configured to automatically adjust light settings of said UV-C light source if said amount of light absorbed is not within a predetermined range.

3. The UV-C based sterilization device of claim 1, wherein said dosing system further comprises a distance sensor;
said distance sensor configured to measure a distance between said UV-C light source and said treatment surface;
wherein said microcontroller is configured to provide notifications if said distance is not within a predetermined range.

4. The UV-C based sterilization device of claim 1, wherein said dosing system further comprises an accelerometer;
said accelerometer configured to measure acceleration at which said UV-C light source moves over said treatment surface;
wherein said microcontroller is configured to provide notifications if said acceleration is not within a predetermined range.

5. The UV-C based sterilization device of claim 1, wherein said dosing system further comprises a speed sensor;
said speed sensor configured to measure a speed at which said UV-C light source moves over said treatment surface;
wherein said microcontroller is configured to provide notifications if said speed is not within a predetermined range.

6. The UV-C based sterilization device of claim 1, wherein said display screen further comprises an indicator light.

7. The UV-C based sterilization device of claim 1, wherein said main console further comprises an image projector that is configured to project an image showing a representation of said treatment surface, further wherein said image is displayed directly on said treatment surface or said display screen.

8. The UV-C based sterilization device of claim 1, wherein said main console further comprises a wireless module for transmitting data to an external device.

9. The UV-C based sterilization device of claim 1, wherein said main console further comprises a timer.

10. The UV-C based sterilization device of claim 1, wherein said main console further comprises speakers.

11. The UV-C based sterilization device of claim 1, wherein said image recognition module is configured to provide an overlaid grid on said three-dimensional mapping of said treatment surface, defining a plurality of grid areas that is displayed on said display screen;
one or more of said plurality of grid areas configured to change colors when said treatment surface correlating to said one or more of said plurality of grid areas is treated.

12. The UV-C based sterilization device of claim 1, wherein said UV-C light source comprises UV-C LED array.

13. The UV-C based sterilization device of claim 1, wherein said UV-C light source comprises a UV-C lamp, further wherein said UV-C lamp comprises a mercury lamp or a xenon lamp.

14. A method of sterilizing skin, the method comprising the steps of:
activating a UV-C based skin sterilizing device, wherein said UV-C based skin sterilizing device comprises:
a main console connected to a delivery system and a display screen, wherein said delivery system comprises a dosing system and a UV-C light source, further wherein said dosing system comprises an image recognition module and a camera, further wherein said display screen comprises a user interface;
capturing images of said treatment site via said camera;
defining a treatment boundary by mapping a treatment site via said image recognition module, wherein said image recognition module is configured to provide a three-dimensional mapping of said treatment surface;
activating said UV-C light source;
delivering UV-C dose on said treatment surface;
calculating said UV-C dose delivered on said treatment surface; and
optimizing said UV-C dose to sterilize said treatment surface.

15. The method of claim 14, further comprising the steps of:
measuring a distance between said treatment site and said UV-C based skin sterilizing device;
if said distance is not within a predetermined range, providing a notification; and
adjusting said distance between said treatment site and said UV-C based skin sterilizing device.

16. The method of claim 14, wherein said image recognition module is configured to provide an overlaid grid on said three-dimensional mapping of said treatment surface, defining a plurality of grid areas that is displayed on said display screen;
one or more of said plurality of grid areas configured to change colors when said treatment surface correlating to said one or more of said plurality of grid areas is treated.

17. The method of claim 16, further comprising the steps of:
tracking said treatment site;
if said treatment site is treated, indicating one or more of said plurality of grid areas corresponding with said treatment site in green on said user interface;
if said treatment site is not treated, indicating one or more of said plurality of grid areas corresponding with said treatment site in red on said user interface.

18. The method of claim 14, further comprising the steps of:
measuring a speed of said UV-C based skin sterilizing device;
if said speed is not within a predetermined range, providing a notification; and
adjusting said speed of said UV-C based skin sterilizing device.

19. The method of claim 14, further comprising the steps of:
measuring acceleration of said UV-C based skin sterilizing device;
if said speed is not within a predetermined range, providing a notification; and
adjusting said acceleration of said UV-C based skin sterilizing device.

20. The method of claim 14, further comprising the steps of:
measuring light absorption on said treatment site;
if said light absorption is not within a predetermined range, providing a notification; and
adjusting a light setting of said UV-C light source via a microcontroller of said main console.
